(19) United States
(12) Patent Application Publication
(10) Pub. No.: US 2007/0099284 A1
(43) Pub. Date: May 3, 2007

(54) Dehalococcoides isolate for bioremediation

(75) Inventor: Frank Loeffler, Atlanta, GA (US)

Correspondence Address: DARBY & DARBY P.C., P. O. BOX 5257, NEW YORK, NY, 10150-5257, US

(73) Assignee: GEORGIA TECH RESEARCH CORPORATION, ATLANTA, GA (US)

(21) Appl. No.: 10/559,993

(22) PCT Filed: Jun. 10, 2004
(86) PCT No.: PCT/US04/19000

§ 371 Date: Dec. 7, 2005

Related U.S. Application Data
(60) Provisional application No. 60/477,799, filed on Jun. 10, 2003.

(51) Int. Cl. A62D 3/02; C12N 1/20

(52) U.S. Cl. 435/252.3; 435/262.5; 588/300; 588/406

(57) ABSTRACT

A bacterium, designated BAV 1 belonging to the Pinellas group within the Dehalococcoides cluster has been discovered. BAV 1 is able to completely metabolize chloroethenes to non-toxic products. The isolate is able to grow with dichloroethenes and vinyl chloride (VC) as electron acceptors, reductively dechlorinating dichloroethenes and VC to ethene and inorganic chloride under anaerobic conditions. The BAV1 isolate is useful for bioremediation approaches for cleaning contaminated subsurface environments and restoring drinking water reservoirs.

### CROSS REFERENCE TO RELATED APPLICATIONS

The present application claims the benefit of priority under 35 U.S.C. §119(e) of provisional application Ser. No. 60/477,799, filed Jun. 10, 2003, which is hereby incorporated herein by reference in its entirety.

### GOVERNMENT SUPPORT

This work was supported in part by the U.S. Department of Defense Strategic Environmental Research and Development Program (contract no. DACA72-00-C-0023) and the National Science Foundation (contract no. IBN-0090496). Accordingly, the United States Government may have certain rights to this invention.

### FIELD OF INVENTION

The invention relates to the bioremediation of toxic compounds. More particularly, the invention relates to methods for the bioremediation of chlorinated environmental pollutants using a novel isolate belonging to the Pinellas group within the Dehalococcoides cluster. The isolate is capable of metabolic (i.e., growth-linked) reduction of dichloroethene isomers, vinyl chloride, and 1,2-dichloroethane.

### BACKGROUND OF THE INVENTION

Concern for environmental safety requires the need to find ways to effectively dispose of hazardous waste and detoxify contaminated sites. Conventional techniques for remediation of toxic waste sites have focused on physical-chemical approaches (e.g., solvent or surfactant flushing, in situ chemical oxidation, excavation, etc.). These techniques often do not transform contaminants into safe products, but instead only contain and/or concentrate the hazardous material. These techniques also will not work at many sites due to the geochemical characteristics and the size of the contaminant plumes. Bioremediation approaches, by contrast, are non-invasive, and offer the potential to convert toxic organic contaminants into end products that are either less toxic or non-toxic. Bioremediation is therefore developing as the method of choice over conventional physical-chemical remediation treatments.

Chlorinated hydrocarbons represent a class of toxic chemicals found frequently at contaminated sites. Bacterial cultures capable of capturing the energy released in reductive dechlorination have been identified, and are attractive candidates for bioremediation of sites contaminated with groundwater pollutants such as, for example, chlorinated ethenes. Tetrachloroethene (a.k.a., PCE, i.e., perchloroethylene) and trichloroethene (TCE) are choice solvents for many industrial applications. The widespread use of these and other solvents has resulted in extensive groundwater contamination. Partial reductive dechlorination of PCE and TCE mediated through abiotic and biotic processes lead to the accumulation of toxic (e.g., dichloroethenes (“DCEs”)) and carcinogenic (e.g., vinyl chloride (“VC”)) intermediates. (Campbell, T. J., et al., (1997) Environ. Toxicol. Chem. 16, 625-630; Allen-King, R. M., et al., Environ. Toxicol. Chem. (1997) 16, 424-429; Abelson, P. H. (1990) Science 250, 733; DiStefano, T. D., et al. (1991) Environ. Microbiol. 57,2287-2292; Freedman, D. L. et al. (1989) Appl. Environ. Microbiol. 55, 2144-2151; Vogel, T. M., et al. (1985) Appl. Environ. Microbiol. 49, 1080-1083; Maymó-Gatell, X., et al. (1995) Appl. Environ. Microbiol. 61, 3928-3933). VC has been found in at least 496 of the 1,430 National Priorities List (NPL) sites identified by the U.S. Environmental Protection Agency (EPA). PCE and TCE are present in at least 771 and 852 NPL sites, respectively (EPA, Agency for Toxic Substances and Disease Registry, ToxFAQs for chlorinated ethenes. (1996; www.atsdr.cdc.gov/tfacts70.html)).

Detoxification of chloroethenes requires complete reductive dechlorination to nonchlorinated end products, such as ethene, and chloride. Substantial information has accumulated describing populations that use polychlorinated ethenes as metabolic electron acceptors (Holliger, C., et al. (1998) FEMS Microbiol. Rev. 22, 383-398; Löffler, F. E., et al., (2003) in Dehalogenation: Microbial processes and environmental applications, Häggblom, M. M. & Bossert, I. D. (eds.), Kluwer Academic Press, New York). To date, however, populations capable of complete reductive dechlorination have remained elusive. Dehalococcoides ethenogenes strain 195 was shown to dechlorinate PCE to VC but failed to grow with VC (Maymó-Gatell, X., et al. (1997) Science 276, 1568-1571). Microbial mineralization of cis-DCE and VC was observed under aerobic conditions (Coleman, N. V., et al. (2002) Appl. Environ. Microbiol. 68, 6162-6171; Coleman, N. V., et al. (2002) Appl. Environ. Microbiol. 68, 2726-2730). DCEs and VC, however, are generated from polychlorinated ethenes in anoxic and reduced environments. Hence, incomplete reductive dechlorination of PCE and TCE remains a problem at many sites and can prevent site restoration. An anaerobic process that leads to complete detoxification is desirable and would be most effective to achieve in situ bioremediation.

The present inventor has discovered a novel Dehalococcoides isolate that uses the products of partial reductive dechlorination, DCEs and VC, as metabolic electron acceptors and, in the process, transforms these toxic compounds into non-toxic end products. The isolate is useful for improved bioremediation approaches to detoxify chloroethene-contaminated aquifers and promote site closures (i.e., complete restoration). The isolate will help promote decontamination of chloroethene-contaminated aquifers at reasonable cost and within acceptable time frames, and to protect threatened drinking water reservoirs.

### SUMMARY OF THE INVENTION

The inventor has discovered a new Dehalococcoides isolate, designated BAV1, that is capable of the complete reduction of halogenated compounds, e.g., DCEs and VC, under anaerobic conditions. Accordingly, in certain embodiments, the invention provides a biologically pure bacterial culture possessing all of the identifying characteristics of Dehalococcoides isolate BAV1.

In other embodiments, the invention provides a pure culture of Dehalococcoides isolate BAV1.

In other embodiments, the invention provides a method of remediating a substrate comprising a halogenated compound, comprising inoculating said substrate with a microorganism possessing all of the identifying characteristics of Dehalococcoides isolate BAV1.

In certain embodiments the aforementioned method provides for remediation of chloroethenes, vinyl halides, and haloalkanes. In certain preferred embodiments the aforementioned method provides for remediation of a dichloroethene or vinyl chloride. In certain embodiments, the dichlorethene is a member selected from the group consisting of cis-DCE, trans-DCE, and 1,1-DCE. In certain embodiments, the vinyl-halide is selected from the group consisting of VC and vinyl bromide.

### BRIEF DESCRIPTION OF THE DRAWINGS

FIG. 1A-C depicts micrographs of isolate BAV1 using (A) epifluorescence and (B-C) scanning electron microscopy.

FIG. 2 depicts Terminal Restriction Fragment Polymorphisms (T-RFLP) digestion profiles of the PCR-amplified 16S rRNA gene from a culture of bacterium BAV1.

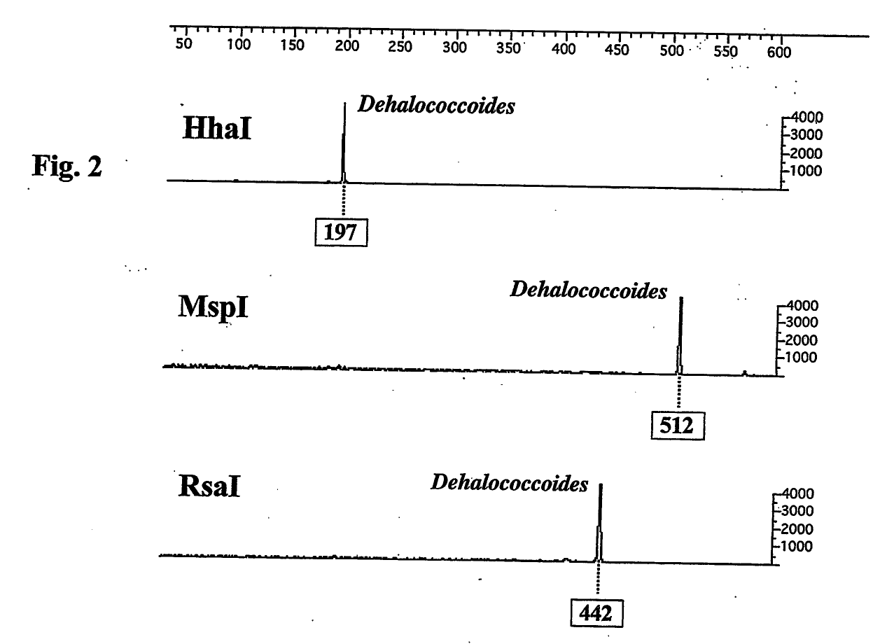

FIG. 3A-B depicts (A) the increase in 16S rRNA gene copies as determined by real-time (RTm) PCR (closed circles) during the reductive dechlorination of VC (closed triangles) to ethene by a culture of bacterium BAV1, and (b) 16S rRNA gene copies of bacterium BAV1 after completely dechlorinating different amounts of VC.

### DETAILED DESCRIPTION OF THE INVENTION

The bacterium BAV1 described herein was isolated from oligotrophic subsurface aquifer material collected inside a chloroethene plume at the Bachman Road Site in Oscoda, Mich. (He, J., et al. (2002) Environ. Sci. Technol. 36, 3945-3952; Sung, Y., et al. (2003) Appl. Environ. Microbiol., 69, 2964-2974). The isolate can be cultured in defined synthetic medium under anaerobic conditions with hydrogen as the electron donor, a dichloroethene or VC as the electron acceptor, and acetate as a source of carbon. PCE and TCE were cometabolized to DCEs in the presence of a growth-supporting chloroethene, and ethene, inorganic chloride, and biomass were the only products formed.

The novel isolate was subjected to various tests and procedures for the determination of identifying characteristics. Examples of assays and procedures for characterizing bacterial isolates include culture-dependent, physiological tests and culture-independent, nucleic acid-based test. The following are non-limiting examples of features characterizing bacterial populations. Other methods for characterizing bacterial isolates are well known in the art.

Culture Medium/Conditions

Pure bacterial cultures may be characterized according to growth in certain types of culture media and conditions. Media may be, e.g., undefined or defined; natural, synthetic or semi-synthetic; and may contain, e.g., a particular carbon source, electron donor, and electron acceptor. Isolates may be characterized as to growth under anaerobic or aerobic conditions and within certain temperature ranges.

The novel Dehalococcoides isolate described herein may be grown in a defined mineral salts medium under anaerobic conditions, as described in Example 1, infra.

Microscopy

Isolates may be characterized using microscopic analysis. Methods of light microscopy include, for example, bright field, phase contrast, differential interference contrast, and Nomarski optics. Isolates may also be characterized using epifluorescence techniques, e.g., of DAPI or acridine orange stained cells. Electron microscopic techniques include transmission and scanning electron microscopy.

Microscopic morphogenic analysis revealed that the Dehalococcoides isolate BAV1 grown in defined medium medium exhibits a disc shape that was distinct from the coccoid morphology of described Dehalococcoides populations (Example 1, infra). BAV1 can also have distinct filamentous appendages (Example 1, infra).

Molecular Analysis

Isolates may be characterized using molecular, nucleic acid-based tools. Isolates may be characterized according to, for example and without limitation, genome size and complexity, or for restriction fragment length polymorphisms (RFLPs) of selected genes. A preferred method of characterization is molecular analysis of ribosomal RNA (rRNA) genes, preferably 16S rRNA genes. 16S rRNA genes may be analyzed using techniques well known in the art, including PCR with universal bacterial pimers or primers specifically targeting 16S rRNA genes of the Dehalococcoides group, RFLP digestion profiles (Amplified Ribosomal DNA Restriction Analysis, ARDRA), Terminal Restriction Fragment Polymorphisms (T-RFLP), and Denaturing Gradient Gel Electrophoresis (DGGE) analysis. Molecular relationships between 16S rRNA gene sequences may be used to infer phylogenetic relationships among bacteria, e.g., between a new isolate and other, known bacteria.

Molecular analysis of 16S rRNA genes of the new isolate BAV1 of the invention is described in Example 2, infra.

Additional Characteristics

Isolates may also be characterized by colony morphology, pleiomorphisms, gram stain and acid fast responses, spore formation, indole, urease and gas production, catalase activity, major and minor fermentation products, secondary fermentation products and phospholipid fatty acid (PLFA) analysis.

Other methods of characterizing bacterial isolates are known in the art. The foregoing methods of characterization are not intended to limit the invention in any way.

The novel bacterial isolate was deposited under the Budapest Treaty with the American Type Culture Collection (ATCC), P.O. Box 1549, Manassas, Va. 20108, USA (www.atcc.org) ______ on and has been assigned Accession Number ______. For the purpose of this invention, any isolate of this bacterium having identifying characteristics of BAV1, including those bacteria having the capability of BAV1 for reducing dichloroethenes and VC to ethene and inorganic chloride under anaerobic conditions, would be effective. Hence, strains having identifying characteristics of BAV1 can be obtained by methods such as, for example and without limitation, mutagenesis, evolution, or mating of strain BAV1.

The bacteria can be mass-produced and maintained for use by any conventional means. The preferred temperature range for growth is 22° C. to about 30° C. and preferred pH range is near pH 7. The bacterial isolate is preferably incorporated into compositions for the desired applications by combining it with a suitable liquid or solid carrier. The actual concentration of BAV1 in the formulated composition is not particularly critical and is a function of practical considerations, such as the type of carrier, the type and amount of toxic substrate to be treated, and the method of application to the substrate.

While not a requisite, the capacity to biotransform greater concentrations and/or amounts of halogenated compounds can be achieved by inclusion of an effective amount of additional agents in the medium. For purposes of formulation and application, an “effective amount” is defined to mean any quantity of the novel Dehalococcoides isolate or compositional adjuvant sufficient to cause partial or complete remediation a target substrate.

Bioremediation may also be performed with a combination of bacterial strains or isolates. Preferred strains useful for bioremediation in combination with isolate BAV1 are bacteria with the ability to directly produce hydrogen or hydrogen precursors during the degradation of fermentable substrates (e.g., lactate, Hydrogen Release Compound (HRC®, available from Regenesis, San Clemente, Calif.), etc.). In certain embodiments, bioremediation can be performed with lactate or HRC® as an electron donor and a combination of BAV1 and fermenting bacteria that produce hydrogen, which serves as source of reducing equivalents to the dechlorinating bacterium BAV1. Bioremediation may also be performed in combination with one or more strain of Dehalococcoides ethenogenes. Strains of Dehalococcoides ethenogenes are described in international publication WO 00/63443.

Isolate BAV1 grows under anaerobic conditions. Hence, it may be desirable to supplement substrates for BAV1 growth and/or remediation with agents that preserve or promote anaerobic conditions. A preferred agent for maintaining anaerobic conditions is HRC®, which is available in an extended release formulation (HRC-X™; Regenesis) adapted for treatment of anaerobically degradable groundwater contaminants. HRC-X™ releases lactate, which is then metabolized by endogenous or supplemented microbes, producing consistent, low-level concentrations of hydrogen. The hydrogen is used by, e.g., BAV1, during reductive dechlorination degradation of DCEs and VC.

Isolate BAV1 has the highly desirable ability to transform toxic halogenated compounds into non-toxic end products. Halogenated compounds include, for example, chloroethenes, vinyl halides, and haloalkanes. Preferred chloroethenes for transformation by BAV1 are VC and DCEs, e.g., cis-DCE, trans-DCE, and 1,1-DCE. Preferred vinyl-halides for transformation by BAV1 are VC and vinyl bromide. A preferred haloalkane is 1,2-dichloroethane.

BAV1 may be used for bioremediation in any contaminated substrate, including, for example and without limitation, contaminated soil, accidental spills, landfill drainages, sediments, aquifers, or water. In a preferred embodiment, a contaminated substrate is a saturated aquifer.

The following examples are intended to further illustrate the invention and are not to be construed as limiting the invention in any way. All patent and literature references cited herein are hereby incorporated in their entireties.

### EXAMPLE 1

### Isolation of BAV1

Bacterium BAV1 was isolated from a PCE-to-ethene-dechlorinating microcosm (He et al. (2002), supra). The microcosms were established with aquifer material from Bachman Road Site aquifer collected inside a chloroethene plume with a Geoprobe at a depth of 18-20 ft below ground surface (He et al (2002), supra). The isolation procedure took advantage of the bacterium's ability to derive all its energy requirements for growth from the reduction of VC to ethene, in a defined synthetic mineral salts medium amended with hydrogen as the electron donor and acetate as a source of carbon. The medium contained per liter: NaCl, 1.0 g; MgCl₂×6 H₂O, 0.5 g; KH₂PO₄, 0.2 g; NH₄Cl, 0.3 g; KCl, 0.3 g; CaCl₂×2 H₂O, 0.015 g; Na₂SO₄, resazurin, 1 mg; trace element solution A, 1 ml; trace element solution B, 1 ml; Na₂S×9 H₂O, 0.048 g; L-cysteine, 0.035 g; NaHCO₃, 2.52 g. Trace element solution A contained per liter: HCl (25% solution, w/w), 10 ml; FeCl₂×4 H₂O, 1.5 g; CoCl₂×6 H₂O, 0.19 g; MnCl₂×4 H₂O, 0.1 g; ZnCl₂, 70 mg; H₃BO₃, 6 mg; Na₂MoO₄×2 H2O, 36 mg; NiCl₂×6 H₂O, 24 mg; CuCl₂×2 H₂O, 2 mg. Trace element solution B contained 6 mg Na₂SeO₃×5 H₂O, 8 mg Na₂WO₄×2 H₂O and 0.5 g NaOH per Reductants were added to the medium after boiling and cooling to room temperature. The headspace was subsequently flushed with oxygen-free H₂/CO₂ (80/20) and the pH was adjusted to 7.2-7.3 by varying the flow of CO₂. The medium was autoclaved before vitamins, hydrogen, and a chloroethene were added. Vitamins were added to a final concentration (in mg per liter) of: biotin, 0.02; folic acid, 0.02; pyridoxine hydrochloride, 0.1; riboflavin, 0.05; thiamine, 0.05; nicotinic acid, 0.05; pantothenic acid, 0.05; vitamin B₁₂, 0.001; p-aminobenzoic acid, 0.05; and thioctic acid, 0.05. (Wolin, F. A., et al. (1963) J. Biol. Chem. 238:2882-2886).

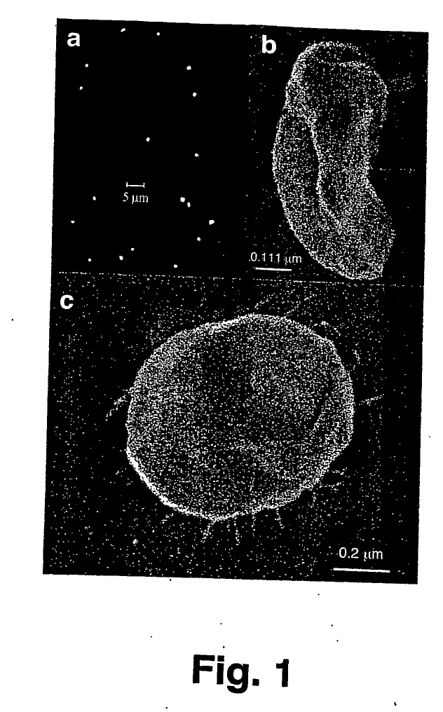

Continued transfers over 4 years in mineral salts medium supplemented with VC, hydrogen, and acetate yielded a nonmethanogenic, ethene-producing culture. Dechlorination occurred with acetate as the sole electron donor, although at lower rates, apparently mediated in association with a syntrophic, acetate-oxidizing partner population (He et al. 2002, 2003). Consecutive transfers without hydrogen achieved further enrichment. VC dechlorination activity was recovered repeatedly from 10⁻⁵ dilutions of consecutive dilution-to-extinction series in hydrogen-amended medium. Following this enrichment procedure, microscopic examination revealed the presence of three morphotypes: a small, disc-shaped organism and two rod-shaped organisms, one short and one long. Initial attempts to obtain the VC-dechlorinating population in pure culture using the dilution-to-extinction principle as well as cultivation in semisolid medium containing 0.5% low melting agarose were unsuccessful. The addition of high concentrations of the peptidoglycan inhibitor ampicillin to cultures, however, did not diminish VC dechlorination activity. Growth in the presence of ampicillin was therefore used to obtain pure cultures of the VC dechlorinating strain. Following five consecutive transfers in liquid medium containing 1 mg ml⁻¹ ampicillin, rod-shaped organisms were no longer detectable in cultures, by microscopic examination. Dechlorinating activity was recovered reproducibly from 10⁻⁷ dilutions in defined completely synthetic medium supplemented with VC, hydrogen and acetate. VC dechlorinating activity was also obtained in cultures inoculated with tiny opaque colonies that developed after 4 to 5 weeks in semisolid medium. Fermentative growth was not observed. The cultures obtained following the foregoing procedures appeared microscopically homogeneous (FIG. 1A). The isolate was designated BAV1 and was deposited with American Type Culture Collection (ATCC), P.O. Box 1549, Manassas, Va. 20108, USA (www.atcc.org) on ______ and assigned accession number ______.

Morphological analysis showed that bacterium BAV1 is a small non-motile organism, approximately 0.8 μm in diameter (FIG. 1A). Light-microscopic analysis suggested that BAV1 cells are disc-shaped, not coccoid like Dehalococcoides ethenogenes and Dehalococcoides sp. strain CBDB1. (Maymó-Gatell (1997), supra; Adrian, L., et al., (2000) Nature 408, 580-583). BAV1 cells suspended in liquid appeared to tumble end over end, a phenomenon explained by a disc-shaped morphology. High-resolution scanning electron microscopy also suggested that BAV1 cells have a disc-shaped rather than a coccoid morphology and feature filamentous appendages on the cell's surface (FIG. 1B and C), which may play a role in adherence and subsequent surface colonization.

The purity of isolate BAV1 was confirmed using 16S rRNA gene-based approaches, including terminal restriction fragment length polymorphism (T-RFLP), denaturing gradient gel electrophoresis (DGGE), and the analysis of 16S rRNA gene clone libraries. The Dehalococcoides ethenogenes strain 195 genome (www.tigr.org) possesses a single ribosomal RNA operon, indicating that the analysis of 16S rRNA genes in Dehalococcoides strains will not be complicated by possible variations in multiple polymorphic 16S rRNA gene sequences. The 16S rRNA gene of isolate BAV1 was amplified by PCR with universal bacterial primers 8F (5′-AGAGTTT GATCCTGGCTCAG-3′; SEQ ID NO: 1) and 1541R (5′-AAGGAGGTGATC CAGCCGCA-3′; SEQ ID NO: 2), which maybe used to amplify 16S rRNA gene sequences from all bacterial strains, and subjected to restriction analysis. The fragment sizes obtained following digestion with HhaI, MspI, and RsaI matched the terminal fragment sizes predicted by in silico digestion of the isolate's 16S rRNA gene sequence. A single 148 bp fragment band, whose sequence exactly matched that of isolate BAV1, was obtained through DGGE analysis. Amplified ribosomal DNA restriction analysis (ARDRA) of 16S rRNA gene clone libraries established with genomic DNA obtained from a VC-grown culture generated patterns predicted by in silico digestion, suggesting that all 16S rRNA gene inserts contained in the clone libraries belonged to isolate BAV1 (data not shown).

Isolate BAV1 grew in defined, completely synthetic mineral salts medium containing acetate, hydrogen, and VC, infra. At 25° C., cultures of BAV1 dechlorinated VC at rates of up to 134.2±10 nmol min⁻¹ mg of protein⁻¹, and grew with a doubling time of 2.2 days to yield 239±27 mg (mean±SD, n=6) of protein per mole of chloride released. Growth of isolate BAV1 depended strictly on reductive dechlorination and the presence of hydrogen as an electron donor, which could not be replaced by organic substrates including formate. VC, cis-DCE, trans-DCE, 1,1-DCE, or 1,2-dichloroethane and vinyl bromide served as growth-supporting electron acceptors for BAV1. In all cases, stoichiometric amounts of ethene accumulated as the reduced end product. Without being bound by theory, the results indicate that two different reductive dechlorination pathways (i.e., dichloroelimination and hydrogenolysis) operate in BAV1, and are involved in the isolate's energy metabolism. Chlorinated compounds that did not support growth of BAV1 included PCE, TCE, chlorinated propanes, 1,1,1-trichloroethane, 1,1-dichloroethane, and chloroethane. PCE and TCE, however, were cometabolized to DCEs (primarily cis-DCE) in the presence of a growth-supporting chloroethene. Since DCEs and VC are intermediates in the reductive dechlorination pathway of PCE and TCE, isolate BAV1 can promote complete reductive dechlorination of PCE and TCE to the environmentally benign end product ethene. Other organic and inorganic electron acceptors tested that were not utilized by BAV1 included nitrate, fumarate, ferric iron, sulfite, sulfate, thiosulfate, sulfur and oxygen.

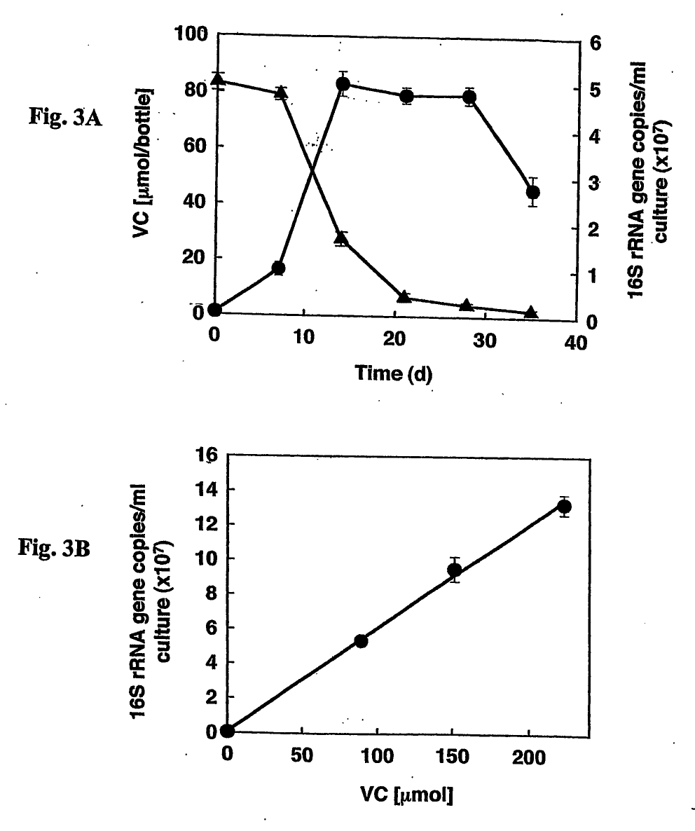

Respiratory growth of isolate BAV1 with DCEs and VC was conclusively demonstrated by the chloroethene-dependent increase in cellular macromolecules (e.g., protein and DNA). Neither cell proliferation nor protein increase was detected in cultures lacking VC, acetate or hydrogen. Real-time (RTm) PCR demonstrated that the increase in cell numbers was concomitant with the consumption of VC (FIG. 3A). A linear increase in biomass (i.e., cells, as measured by the increase of 16S rRNA gene copies) occurred with increasing amounts of VC provided as electron acceptor, indicating a tight coupling between reductive dechlorination and growth (FIG. 3B). The number of 16S rRNA gene copies measured in cultures without VC corresponded to the number of cells transferred with the inoculum, indicating that no growth occurred in the absence of VC. Cultures that had consumed 80 μmoles of cis-DCE contained about twice as many 16S rRNA gene copies as cultures grown with 80 μmoles of VC (e.g., 9.28±0.41 versus 4.99±0.26×10⁷ copies). These findings demonstrated that BAV1, when grown with cis-DCE, captured energy from both dechlorination steps.

### EXAMPLE 2

### Phylogenetic Analysis of BAV1

Phylogenetic analysis carried out with double stranded DNA sequencing of BAV1 16S rRNA gene demonstrated that the VC-dechlorinating isolate belonged to the Pinellas group within the Dehalococcoides cluster (Hendrickson, E. R., et al. (2002) Appl. Environ. Microbiol. 68,485-495), which includes strains CBDB1 (AF230641) and FL2 (AF357918.2). Table 1 displays known metabolic electron acceptors along with phylogenetic grouping of known Dehalococcoides-like populations. BAV1 shared a highly similar or identical 16S rRNA gene sequence with Dehalococcoides populations that lack the capacity to dechlorinate chloroethenes or grow with VC as a metabolic electron acceptor. The high degree of 16S rRNA gene sequence similarity among members of the Dehalococcoides cluster implies that analysis of 16S rRNA gene sequences cannot distinguish between populations of this group exhibiting different physiological activities. Hence, focusing on 16S rRNA gene sequence analysis is not sufficient for characterizing the dechlorinating community or to reliably predict the dechlorination potential associated with a particular environment.

TABLE 1
16S rRNA gene sequence similarities and electron acceptor utilization
profiles of Dehalococcoides spp.^{a}

| | | Percent Similarity | | | | | |
|---|---|---|---|---|---|---|---|
| | | (no. of base differences) | | | | | |
| Dehalococcoides sp. | Electron acceptors | (1) | (2) | (3) | (4) | (5) | (6) |
| (1) Dhc. ethenogenes | PCE, TCE, 1,2- | — | 98.2 | 98.1 | 98.2 | 97.1 | 87.0 |
| strain 195^{e} (C^{b}) | cis-DCE, 1,1-DCE, | | (21) | (22) | (21) | (25) | (154) |
| | dichloroethane, | | | | | | |
| | 1,2-dibromoethane | | | | | | |
| (2) Dhc. sp. strain | TCE, cis-DCE, | | — | 99.9 | 100 | 99.7 | 87.3 |
| FL2^{f} (P^{b}) | trans-DCE | | | (1) | (0) | (4) | (150) |
| (3) Dhc. sp. strain | cis-DCE, trans-DCE, | | | — | 99.9 | 99.7 | 87.2 |
| BAV1^{g} (P^{b}) | 1,1-DCE, VC, vinyl | | | | (1) | (3) | (151) |
| | bromide, 1,2-dichloroethane | | | | | | |
| (4) Dhc. sp. strain | 1,2,3-TCB^{c}, 1,2,4-TCB | | | | — | 99.7 | 87.3 |
| CBDB1^{h} (P^{b}) | 1,2,3,4-TeCB^{c}, 1,2,3,5-TeCB, | | | | | (4) | (150) |
| | 1,2,4,5-TeCB | | | | | | |
| (5) Dhc. sp. 1,2-Dⁱ (P^{b}) | 1,2-dichloropropane | | | | | — | 87.2 |
| | | | | | | | (154) |
| (6) Bacterium DF-1^{j, k} | polychlorinated biphenyls | | | | | | — |
| | with doubly flanked chlorines, | | | | | | |
| | hexachlorobenzene | | | | | | |

^{a}The percent similarity calculations were based on the alignment of 1181 nucleotide positions.
^{b}Group designations according to Hendrickson, E. R., et al. (2002) Appl. Environ. Microbiol. 68, 485-495; C/Cornell, P/Pinellas sequence subgroups
^{c}TCB, trichlorobenzene; TeCB, tetrachlorobenzene.
^{d}The 16S rRNA gene sequence of the 1,2-D-dechlorinating population is identical to that of BAV1 through identified Dehalococcoides signature regions (Hendrickson et al., supra).
^{e}Genbank Accession no. AF004928
^{f}Genbank Accession no. AF357918
^{g}Genbank Accession no. AY165308
^{h}Genbank Accession no. AF230641
ⁱGenbank Accession no. AY185324
^{j}Genbank Accession no. AY393781
^{k}Wu, Q., et al. (2002) Environ. Sci. Technol 36, 3290-3294

### EXAMPLE 3

### Biotransformation of PCE-Contaminated Aquifer Samples

PCE-contaminated aquifer material from a chloroethene-impacted site in Michigan (Bachman Road Site) (Lendvay et al. (2003) Environ. Sci. Tech., 37, 1422-1431) was tested to determine whether bioaugmentation with a mixed culture containing BAV1 promoted complete detoxification to ethene. Aquifer materials from the Bachman Road site were retrieved from the saturated zone inside the chloroethene plume with a Geoprobe. Procedures for sampling and microcosm setup were essentially performed as described (Fennell et al. 2001, He et al. 2002). The cores were capped and immediately stored at 4° C. Inside an anaerobic chamber (Coy, Ann Arbor, Mich.) filled with H₂/N₂ (3%/97% [vol/vol]), the aquifer material was transferred to sterilized glass jars (1 liter volume), and thoroughly mixed. Aquifer material (40 g, wet weight) was placed into sterile 160-ml serum bottles. Sterile, O₂-free, phosphate buffer (10 mM, pH 7.2) or anoxic groundwater collected at the sampling location supplemented with 5 mM acetate was added to the bottles before they were sealed with butyl rubber stoppers (Geo-Microbial Technologies, Inc., Ochelata, Okla.). Hydrogen and 0.1 mM PCE were added to the bottles. To prevent contamination, aseptic technique was applied to the greatest extent possible during sample collection, material handling, and microcosm setup inside the anaerobic chamber. Microcosms were incubated stationary at 25° C. in the dark. Chloroethenes were measured with a Hewlett Packard model 6890 gas chromatograph equipped with a HP-624 column (60 m length, 0.32 mm diameter, 1.8 μm film thickness) and a flame ionization detector (FID). Headspace samples (100 or 200 μl) were withdrawn with gas-tight 250 μl glass syringes (model #1725, Hamilton Co., Reno, Nev.), and manually injected into a split injector operated at a split ratio of 2:1.

Microcosms that initially showed no or incomplete reductive dechlorination of PCE were inoculated with 2% (vol/vol) of the BAV1-containing enrichment culture grown with cis-DCE and lactate. Only the microcosms supplemented with BAV1 converted chloroethenes to stoichiometric amounts of ethene, after a 2-month incubation period. Microcosms that did not receive the BAV1 inoculum failed to produce ethene.

A field test bioaugmentation study in a hydraulically controlled recirculation test plot (4.6 m×5.5 m) at the Bachman site validated that bioaugmentation with BAV1 can be used for detoxification at sites where the in situ microbiology to drive the reductive dechlorination process to completion is absent or the rates for contaminant removal are insufficient (Lendvay et al., supra; He, J., et al. (2003) Appl. Environ. Microbiol., 69, 996-1003).

### EXAMPLE 4

### Methods

Medium Preparation

A completely defined, anaerobic mineral salts medium (He et al. (2003), supra; Löffler, F. E., et al. (1997) Appl. Environ. Microbiol. 63, 4982-4985; Löffler, F. E., et al. (1999) Appl. Environ. Microbiol. 65, 4049-4056) was prepared as described in Example 1, supra. The enrichment and isolation process was carried out in 20-ml glass vials containing 10 ml (final volume) of growth medium, while the cultures used for kinetic studies or molecular analyses were executed in 160-ml serum bottles containing 100 ml of medium. Unless indicated otherwise, cultures were incubated at 30° C. in the dark without shaking. Soluble substrates were added at 5 mM. Hydrogen was added by syringe at twice the concentration required for complete reductive dechlorination (i.e., 8.5-17 kPa). Chlorinated compounds were added to final aqueous concentrations ranging from 0.47 to 1.33 mM. Semisolid medium was prepared by adding 0.5% (weight by weight) low melting agarose before autoclaving.

Analytical Methods Protein content of liquid cultures was estimated as follows: Cells were harvested from 8 ml of culture fluid by centrifugation (10 min, 10,000 g), and following alkaline cell lysis (Gerhardt, P. et al. (eds.) (1981) Manual of methods for general bacteriology (American Society for Microbiology, Washington, D.C.), the Coomassie plus protein assay reagent kit (Pierce Inc., Rockford, Ill.) was used according to the manufacturer's recommendations. Spectrophotometric determination at 595 nm was used to quantify protein by comparing the sample absorbance with protein standards of known concentration prepared the same way as the samples. Chloroethene and ethene concentrations were determined by gas chromatography as described (He et al. (2003), supra).

Electron Microscopy

A Zeiss LSM 510 confocal microscope with a Plan-Neofluar objective (100×/1.3) was used to obtain micrographs of cell suspensions following acridine orange staining (15-60 m inn 0.01% aqueous solution). Scanning electron microscopy (SEM) micrographs were obtained using the TOPCON DS-130 field emission SEM with samples staged “in-lens” and photographed at 20 kilovolts. Samples were prepared as described (Sung et al., supra), and coated with chromium (about 1.5 nanometer thickness) in a Denton DV-602 turbo magnetron sputter system.

Molecular Analyses

DNA was extracted from actively growing culture as described by He et al. (2003), supra. Real-time PCR to quantify isolate BAV1 cells utilized a Dehalococcoides-16S rRNA gene-targeted probe tagged with a 6-carboxy-fluorescein (FAM) reporter fluorochrome on the 5′ end, and N,N,N′,N′-tetramethyl-6-carboxy-rhodamine (TAMRA) quencher on the 3′ end as described previously (He et al. (2003), supra). Linear calibration curves (r²>0.99) were generated spanning a template concentration range from 6.9×10² to 6.9×10⁶ 16S rRNA gene copies per 30-μl reaction volume using BAV1 genomic DNA or plasmid DNA containing BAV1's 16S rRNA gene. Analysis of individual clones with ARDRA was performed as described before (He et al. (2003); Löffler, F. E., et al. (2000) Appl. Environ. Microbiol. 66, 1369-1374), except that the PCR amplified 16S rDNA products from each clone were digested with enzymes HhaI, MspI and RsaI for 3h at 37° C. The reactions were terminated by incubation at 60° C. for 10 min according to the manufactures recommendations (Gibco), and the resulting fragments were resolved on 2.5% low melting agarose gel (FMC, Seaplaque) for 2h. The fluorescently labelled primer 8F (5′-AGAGTTTGATCCTGG CTC AG-3′; SEQ ID NO: 1) and unlabeled 1492R (5′-GC(C/T)TACCTTGTTACGAC TT-3′; SEQ ID NO: 3) were used to obtain a fluorescently labelled 16S rRNA gene amplicons from pure culture DNA. Fluorescently labeled terminal fragments obtained by digesting the amplicon with HhaI, MspI and RsaI were analyzed at Michigan State University's Genomics Technology Support Facility. DGGE analysis was performed by Microbial Insights Inc. (Rockford, Tenn.) using universal bacterial primers corresponding to E. coli positions 341-534 (Muyzer, G., et al. (1993) Appl. Environ. Microbiol. 59, 695-700).

What is claimed:

1. A biologically pure bacterial culture possessing all of the identifying characteristics of Dehalococcoides isolate BAV1.

2. The biologically pure culture claim 1, which is isolate BAV1.

3. A method of remediating a substrate comprising a halogenated compound, said method comprising inoculating said substrate with a microorganism possessing all of the identifying characteristics of Dehalococcoides isolate BAV1.

4. The method of claim 3 wherein said halogenated compound is a member selected from the group consisting of chloroethenes, vinyl halides, and haloalkanes.

5. The method of claim 4 wherein said halogenated compound is a dichloroethene or vinyl chloride.

6. The method of claim 5 wherein said dichlorethene is a member selected from the group consisting of cis-DCE, trans-DCE, and 1,1-DCE.

7. The method of claim 5 wherein said halogenated compound is VC.

8. The method of claim 4 wherein said vinyl-halide is selected from the group consisting of VC and vinyl bromide.

9. A method of remediating a substrate comprising a halogenated compound, said method comprising inoculating said substrate with a microorganism that is Dehalococcoides isolate BAV1.

10. The method of claim 9 wherein said halogen-compound is a member selected from the group consisting of chloroethenes, vinyl halides, and haloalkanes.

11. The method of claim 10 wherein said halogenated compound is a dichloroethene.

12. The method of claim 11 wherein said dichlorethene is a member selected from the group consisting of cis-DCE, trans-DCE, and 1,1-DCE.

13. The method of claim 11 wherein said halogenated compound is VC.

14. The method of claim 9 wherein said vinyl-halide is a member selected from the group consisting of VC and vinyl bromide.


SEQUENCE LISTING (3 sequences)

SEQ ID NO: 1 - DNA, 20 bp, artificial sequence
  primer
agagtttgatcctggctcag

SEQ ID NO: 2 - DNA, 20 bp, artificial sequence
  primer
aaggaggtgatccagccgca

SEQ ID NO: 3 - DNA, 19 bp, artificial sequence
  primer
  misc_feature (3)..(3): n is c or t
gcntaccttgttacgactt